(19) United States
(12) Patent Application Publication
(10) Pub. No.: US 2021/0186714 A1
(43) Pub. Date: Jun. 24, 2021

(54) FOOT SHELL SYSTEM

(71) Applicant: OTTOBOCK SE & CO. KGAA, Duderstadt (DE)

(72) Inventor: Markus SCHNEEGANS, Rollshausen (DE)

(73) Assignee: OTTOBOCK SE & CO. KGAA, Duderstadt (DE)

(21) Appl. No.: 17/124,423

(22) Filed: Dec. 16, 2020

(30) Foreign Application Priority Data
Dec. 18, 2019 (DE) 10 2019 134 984.2

(51) Int. Cl. A61F 2/66

(52) U.S. Cl. CPC A61F 2/66; A61F 2002/5001

(57) ABSTRACT

A system with a foot shell, which has a forefoot inner contour and a heel inner contour, at least two prosthetic feet, each with a forefoot region and a heel region, and at least two forefoot inserts that can be positioned at the forefoot region of at least one prosthetic foot, and/or at least two heel inserts that can be positioned at the heel region of at least one prosthesis region, wherein each prosthetic foot has an outer contour of the forefoot with at least one forefoot insert positioned at its forefoot region, which is adapted to the forefoot inner contour of the foot shell, and/or that each prosthetic foot with at least one heel insert positioned at its heel region has a heel outer contour which is adapted to the heel inner contour of the foot shell.

The invention relates to a system with a foot shell with a forefoot inner contour and a heel inner contour, at least two prosthetic feet, each with a forefoot region and a heel region, and at least two forefoot inserts that are positioned at the forefoot region of at least one prosthetic foot, and/or at least two inserts that can be positioned at the heel region of at least one prosthesis region.

This type of system has been known within the scope of the prior art for many years. The prosthetic foot itself, for example, has several leaf springs that provide the required elasticity, for example during heel strike or push-off by the toes. In addition, the prosthetic foot usually has a fixing device with which another prosthetic element, for example a lower leg tube, can be attached to the prosthetic foot. Such a prosthetic foot therefore provides the basic functionality of the foot, but is usually not individually adapted to the wearer of the prosthetic foot.

To change this, for example, a prosthetic foot is known from DE 10 2014 006 687 A1, in which a receiving frame can be arranged in the forefoot area. This section, also known as the tongue, may exhibit a different hardness, stiffness and/or elasticity than the rest of the prosthetic foot and thus change the rolling behaviour of the prosthetic foot.

Together with this receiving frame, the prosthetic foot is then arranged in the foot shell, which is made of a plastic material, for example, to lend the prosthetic foot a visual appearance similar to that of a healthy foot. Depending on the prosthetic foot used, which may be designed differently for different requirements, different receiving frames must be arranged in the forefoot region to adapt the function of the prosthetic foot to the individual needs of the wearer. This means that different foot shells have to be used, as the spatial extensions of the combination of prosthetic foot and receiving frame change, for example.

U.S. Pat. No. 8,128,709 B2 describes a system in which additional elements, such as shock-absorbing elements, energy-storing elements and elements that change the stiffness and strength of the foot are arranged in the foot shell.

US/2015/0374514 A1 describes prosthetic feet in which the leaf spring forming the foot part is flattened on the lower side in the toe region so that forefoot elements can be arranged at this point. These allow the mechanical properties of the foot in the forefoot region to be adapted to the needs of the wearer. This combination can be introduced into individually designed shells.

WO 2014/126768 A1 describes the attachment of forefoot elements to a prosthetic foot, which are preferably detachably connected to the rest of the foot, preferably to a sole element. These elements change the shape of the sole element as the element protrudes beyond the contour of the sole element. This allows the shape to be approximated to the shape of a natural foot. A foot shell that surrounds such a foot is adapted to the overall shape of the foot.

The number of parts required, for example by the orthopedic technician in order to provide the optimum prosthesis for as many patients as possible, is therefore extremely high. In addition, it is difficult to replace and exchange individual elements, as this also entails the replacement of other components. If, for example, it has become apparent over the course of time that a receiving frame used is too soft for the wearer of the prosthesis and must be replaced with a harder one, this also involves an exchange of the foot shell, since the spatial extension of the prosthetic foot changes with the receiving frame attached to it.

Another disadvantage of foot shells known from the prior art is that they are adapted to one prosthetic foot. If an individual prosthetic foot is subsequently modified or if changes are made to the entire model range, the foot shell must also be readjusted. New tools are therefore also required for the production of of the foot shell, which means a considerable additional expenditure of time and money. Furthermore, foot shells for different prosthetic feet are not interchangeable. An orthopedic technician must therefore also provide a suitable foot shell for each prosthetic foot.

The invention therefore aims to improve a system of the type mentioned above in such a way that the number of parts required is reduced and adaptation to individual conditions or to changes in the prosthetic foot is easier.

The invention solves the problem by way of a system according to the generic term in claim 1, which is characterized by the fact that each prosthetic foot has a forefoot outer contour with at least one forefoot insert positioned at its forefoot region, which is adapted to the forefoot inner contour of the foot shell, and/or that each prosthetic foot with at least one heel insert positioned at its heel region has a heel outer contour which is adapted to the heel inner contour of the foot shell.

With such a system, the number of foot shells required is significantly reduced because it preferably comprises at least one insert for each prosthetic foot, wherein said insert is designed in such a way that the common outer contour is adapted to the respective inner contour of the foot shell. If, for example, a prosthetic foot is replaced because it has turned out to be less than ideal, it is not necessary to change the foot shell as well, since there is also a forefoot insert and/or heel insert for the new prosthetic foot that is replacing the previous one, which ensures that the respective outer contour of prosthetic foot and insert is adapted to the respective inner contour of the foot shell. If a prosthetic foot is reworked or a new prosthetic foot is to be offered, it is now no longer necessary to adjust the foot shell and the tools required for production. Only the respective insert, i.e. forefoot insert and/or heel insert, must be adapted to the new or modified prosthetic foot. Changes to existing prosthetic feet, such as new generations of existing products, are thus made more easily, quickly and cost-effectively. Moreover, especially for small and very small quantities, the respective inserts can also be produced in manufacturing processes for which no expensive tools are required, for example by means of additive manufacturing processes such as 3-D printing.

In an especially preferred embodiment of the system, the forefoot inserts and the prosthetic feet are designed so that each combination of a prosthetic foot and a forefoot insert results in a common forefoot outer contour, which is adapted to the forefoot inner contour of a single foot shell and/or in such a way that the heel outer contour is adapted to the heel inner contour in such a way that the prosthetic foot is supported medially and laterally on the foot shell with the heel insert positioned at its heel region. Alternatively, it can be an advantage that the respective common outer contour is adapted to the respective inner contour of, for example, three foot shells which differ only in size, for example.

The forefoot outer contour is preferably adapted to the forefoot inner contour in such a way that the prosthetic foot is supported medially and laterally by the forefoot insert positioned at its forefoot region. Alternatively or additionally, the heel outer contour is preferably adapted to the heel inner contour in such a way that the prosthetic foot is supported medially and laterally by the heel insert positioned at its heel region. This means that a displacement of the prosthetic foot and/or the respective insert medially and laterally relative to the foot shell is not possible if the prosthetic foot with the insert is included in the foot shell. This can also inhibit movement in other directions.

Preferably, the forefoot outer contour is preferably adapted to the forefoot inner contour in such a way that the prosthetic foot is frontally supported by the forefoot insert positioned at its forefoot region. Alternatively or additionally, the heel outer contour is preferably adapted to the heel inner contour in such a way that the prosthetic foot is supported medially and laterally by the heel insert positioned at its heel region. This means that a displacement of the prosthetic foot and/or the forefoot insert to the front, i.e. forward towards the forefoot region, and/or a displacement of the prosthetic foot and/or the heel insert to the rear, i.e. towards the heel region, relative to the foot shell is not possible if the prosthetic foot with the respective insert is included in the foot shell.

It is especially preferred if the forefoot outer contour, at least in sections, but especially preferred completely, is designed in such a way that it corresponds to the forefoot inner contour. Alternatively or additionally, the heel outer contour preferably corresponds, at least in sections, but preferably completely, to the heel inner contour. If the prosthetic foot with the corresponding insert is included in the foot shell, the respective outer contour in this configuration rests at least partially, but preferably completely, against the respective inner contour of the foot shell.

In a preferred configuration of the system, the forefoot inserts and/or heel inserts can be attached to the respective prosthetic foot and/or foot shell, preferably such that they can be detached. This can be achieved, for example, by means of positive-locking elements, where in the case of a forefoot insert, for example, corresponding positive-locking elements are arranged at the heel end of the forefoot insert and at the forefoot end of the prosthetic foot. In a heel insert, the corresponding positive-locking elements are arranged at the forefoot end of the heel insert and at the heel end of the prosthetic foot. These can be clips and snap elements, press studs or velcro elements. It is particularly preferable if all prosthetic feet of the system and all forefoot inserts and/or all heel inserts of the system feature such correspondingly designed positive-locking elements, so that each of the forefoot inserts and/or each heel insert can be arranged on each of the prosthetic feet. Alternatively or additionally, the respective inserts can be fixed to the inside of the foot shell. This can also be achieved using correspondingly designed positive-locking elements. Also in this case, it is advantageous if the positive-locking elements arranged on the inside of the foot shell correspond to the positive-locking elements arranged on all forefoot inserts and/or on all heel inserts. It has proved to be particularly advantageous if the forefoot inserts and/or the heel inserts have, on the one hand, positive-locking elements corresponding to positive-locking elements arranged on the prosthetic feet and, on the other hand, positive-locking elements corresponding to positive-locking elements arranged inside the foot shell.

Alternatively, it can be advantageous to permanently bond the forefoot and/or heel inserts to the foot shell, for example by gluing them in. In many cases, after selecting the prosthetic foot and foot shell, no further changes are made and the foot shell applied. In these cases, the undetachable connection may require less production effort than a detachable form of fixing. Nevertheless, even in this case the system offers the advantage that an orthopedic technician only needs to stock one type of foot shell, possibly in different sizes, and only this one type of foot shell needs to be produced. The associated forefoot insert and/or heel insert can, for example, be permanently connected to the foot shell by the orthopedic technician after the prosthetic foot has been selected.

It is particularly preferable if the heel outer contour, which is formed by the heel region of the prosthetic foot and the heel insert arranged thereon, is designed to match the heel inner contour of the foot shell, regardless of which prosthetic foot and which heel insert is used. It can be advantageous to use several, for instance three, different foot shells that differ in size, for example.

Preferably the heel inserts and/or forefoot inserts differ in their hardness, elasticity and/or flexibility. It can certainly be an advantage if the hardness, elasticity and/or flexibility of the heel inserts and/or forefoot inserts is not constant but varies. This can be achieved, for example, by having a heel insert and/or forefoot insert made of different materials with different degrees of hardness, elasticity and/or flexibility. By distributing the different materials within a heel insole and/or forefoot insole, the functionality of the respective insole and the entire prosthetic foot can be adapted in detail to the individual needs of individual wearers of the prosthesis. In particular, the damping properties of the prosthetic foot can be adjusted through the selection of the inserts.

In a preferred configuration, at least one forefoot insert has several articulated elements. Preferably two, especially preferably three or four such elements are articulated. Although it is particularly preferable if the elements are movable relative to each other, and particularly preferable if they can be swivelled relative to each other about a swivel axis in each case, the movement is damped, i.e. particularly hindered by a damping element, for example a passive actuator, such as a spring element or an elastic element. These actuators, damping elements or spring elements can also be arranged interchangeably on other components of the respective insert, so that individual adjustability is also achieved here.

Preferably at least one forefoot insert features at least one actuator, in particular at least one spring element, and/or at least one deceleration element and/or at least one sensor, in particular at least one pressure sensor and/or at least one acceleration sensor.

The heel inserts of the system preferably have different heel heights.

The inserts are preferably provided with material recesses, for example in the form of grooves. This enables material and thus weight to be saved. Additional functions are also achieved by way of the positioning and/or shape of the material recesses. For example, transverse grooves on the forefoot insert increase flexibility during dorsal flexion and/or plantar flexion. Longitudinal grooves, on the other hand, increase flexibility in the medial and lateral direction.

The system preferably features at least one overall insert, which is made up of a forefoot insert and a heel insert, which are designed to be single-piece. Preferably, the forefoot insert and the heel insert are connected via a connecting element that extends along the sole of the prosthetic foot in a particularly preferable embodiment.

It is especially preferable if the system feature several such overall inserts.

In the following, some examples of embodiments of the present invention will be explained in more detail by way of the attached figures: They show

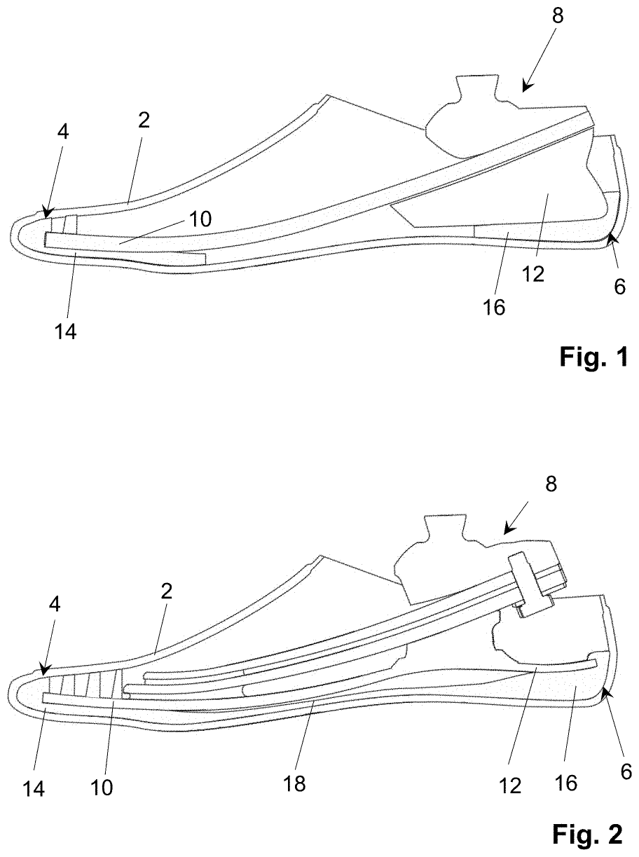

FIG. 1 the schematic sectional representation through a part of a system according to a first example of an embodiment of the present invention,

FIG. 2 the schematic sectional representation through a part of another system,

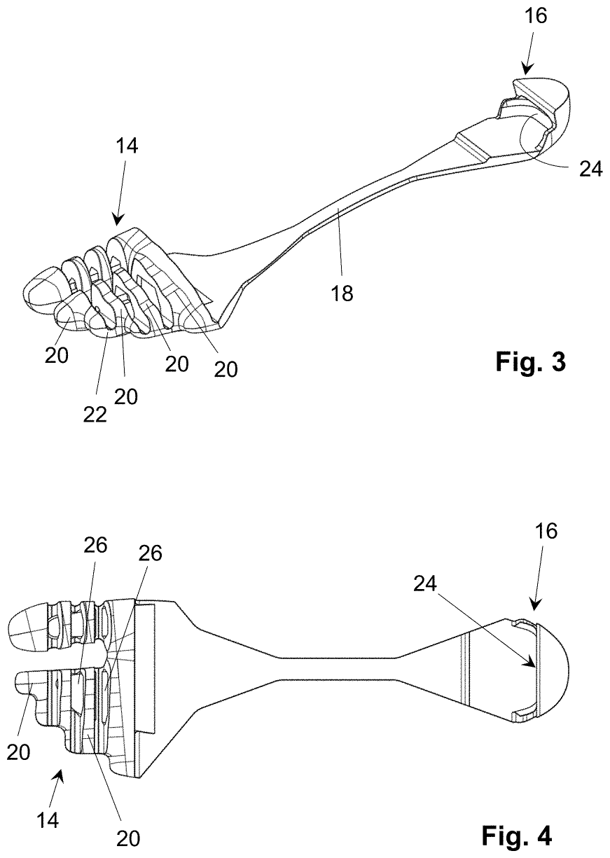

FIG. 3 the schematic three-dimensional view of an overall insert,

FIG. 4 the overall insert from FIG. 3 in another view, and

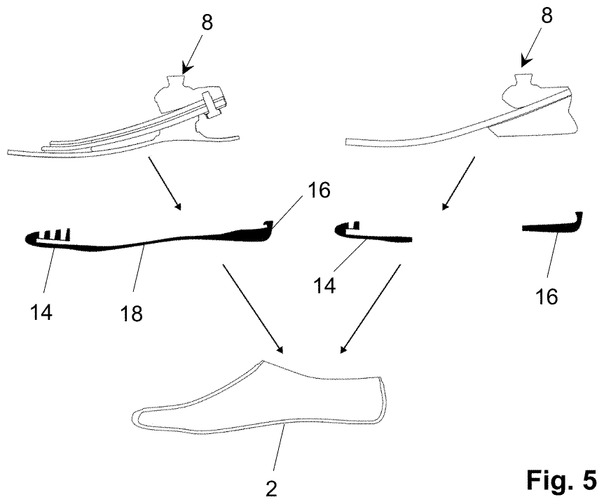

FIG. 5 the schematic representation showing the operating principle of a system according to an example of an embodiment of the present invention.

FIG. 1 schematically depicts the sectional representation through a part of a system according to a first example of an embodiment of the present invention. One can see a foot shell 2, which has a forefoot inner contour 4 and a heel inner contour 6. A prosthetic foot 8 is arranged within the foot shell 2, wherein said prosthetic foot has a forefoot region 10 and a heel region 12. A forefoot insert 14 is arranged at the forefoot region 10, said insert having a forefoot outer contour that is adapted to the forefoot inner contour 4 of the foot shell 2. There is a heel insert 16 at the heel region 12 of the prosthetic foot 8, wherein the heel outer contour of said insert interacts with the heel inner contour 6 of the foot shell 2.

FIG. 2 schematically depicts a similar representation. However, a different prosthetic foot 8 is now placed within the foot shell 2, said prosthetic foot in particular having a differently shaped forefoot region 10 and a differently shaped heel region 12. However, the forefoot inner contour 4 as well as the heel inner contour 6 of the foot shell 2 correspond to the configuration shown in FIG. 1. Therefore, there is a forefoot insert 14 between the forefoot region 10 of the prosthetic foot 8 and the inner contour of the forefoot 4, said insert being different from FIG. 1. The heel insert 16 used also differs from the heel insert 16 in FIG. 1, as the inner contour of the heel insert 16 is matched to the heel region 12 of the prosthetic foot 8 used in FIG. 2. The heel outer contour of the heel insert 16 is again matched to the heel inner contour 6 of the foot shell 2. In the example of an embodiment shown, the forefoot insert 14 is designed as a single piece with the heel insert 16. A connecting element 18 extends underneath the sole of the prosthetic foot 8 and connects the two inserts to each other so that they form a common overall insert.

FIG. 3 shows such an overall insert in a schematic three-dimensional view. The connecting element 18, which connects the forefoot insert 14 with the heel insert 16, is clearly recognizable. The forefoot insert 14 features several articulated elements 20. In this way, in the example of the embodiment shown, a rolling movement can be adapted to that of a healthy foot. Between each of the individual elements 20 is a film hinge 22, which is achieved by the thinning of the material shown.

FIG. 4 depicts a top view of the overall insert from FIG. 3. In the example of an embodiment shown, the heel insert 16 has an undercut 24 to connect it to a heel region 12 of a prosthetic foot 8. The forefoot insert 14 again features the articulated elements 20, between which there are openings 26. The sensitivities and flexibility of the components and elements used are changed through these openings 26 and can thus be adapted to the individually desired properties.

FIG. 5 schematically depicts the different structures shown in the previous figures. The two different prosthetic feet 8 are each fitted with a forefoot insert 14 and a heel insert 16 before they are placed in the foot shell 2. While in the right-hand part of figure 5 the forefoot insert 14 and the heel insert 16 are two separate components, in the left-hand part of FIG. 5 they are connected via a connecting element 18 and thus form a single-piece overall insert.

### REFERENCE LIST

2 foot shell
4 forefoot inner contour
6 heel inner contour
8 prosthetic foot
10 forefoot region
12 heel region
14 forefoot insert
16 heel insert
18 connecting element
20 element
22 film hinge
24 undercut
26 opening

1. A system comprising:
a foot shell, which has a forefoot inner contour and a heel inner contour;
at least two prosthetic feet, each with a forefoot region and a heel region;
at least one of two or more forefoot inserts that can be positioned at the forefoot region of at least one prosthetic foot, or at least two heel inserts that can be positioned at the heel region of at least one prosthesis region;
wherein at least one of each prosthetic foot has an outer contour of the forefoot region with at least one forefoot insert positioned at the forefoot region, the outer contour being adapted to the forefoot inner contour of the foot shell, or each prosthetic foot with at least one heel insert positioned at its heel region has a heel outer contour which is adapted to the heel inner contour of the foot shell.

2. The system according to claim 1, wherein at least one of the forefoot outer contour is adapted to the forefoot inner contour in such a way that the prosthetic foot is supported medially and laterally on the foot shell by the forefoot insert positioned on its forefoot region, or the heel outer contour is adapted to the heel inner contour in such a way that the prosthetic foot is supported medially and laterally on the foot shell by the heel insert positioned on its heel region.

3. The system according to claim 1, wherein at least one of the forefoot outer contour is adapted to the forefoot inner contour in such a way that the prosthetic foot is supported frontally on the foot shell by the forefoot insert positioned on its forefoot region, or the heel outer contour is adapted to the heel inner contour in such a way that the prosthetic foot is supported on the foot shell at the rear by the heel insert positioned on its heel region.

4. The system according to claim 1, wherein at least one of the forefoot outer contour, at least in sections, corresponds to the forefoot inner contour, or the heel outer contour, at least in sections, corresponds to the heel inner contour.

5. The system according to claim 1, wherein at least one of the forefoot inserts or the heel inserts can be attached to at least one of the respective prosthetic foot or in the foot shell.

6. The system according to claim 1, wherein at least one of the heel inserts or the forefoot insert exhibit different degrees of at least one of hardness, elasticity or flexibility.

7. The system according to one of the preceding claims, characterized in that claim 1, wherein at least one forefoot insert comprises several articulated elements.

8. The system according claim 1, wherein at least one forefoot insert comprises at least one of at least one actuator, at least one deceleration element, or at least one sensor.

9. The system according to claim 1, wherein the heel inserts of the system have different heel heights.

10. The system according to claim 1, wherein the system comprises at least one overall insert, formed of a forefoot insert and a heel insert, which are designed to be single-piece.

11. The system according to claim 10, wherein the system comprises several overall inserts.

12. A system comprising:
a foot shell having a forefoot inner contour and a heel inner contour;
at least two prosthetic feet, each having a forefoot region and a heel region, the forefoot region having an outer contour;
at least one of
two forefoot inserts positioned at the forefoot region of at least one of the prosthetic feet, or
at least two heel inserts positioned at the heel region of at least one of the prosthetic feet;
wherein at least one of
each prosthetic foot has at least one forefoot insert positioned at the forefoot region, the outer contour being adapted to the forefoot inner contour of the foot shell, or
each prosthetic foot with at least one heel insert positioned at its heel region has a heel outer contour which is adapted to the heel inner contour of the foot shell.

13. The system according to claim 1, wherein at least one of the forefoot outer contour is adapted to the forefoot inner contour in such a way that the prosthetic foot is supported medially and laterally on the foot shell by the forefoot insert positioned on its forefoot region, or the heel outer contour is adapted to the heel inner contour in such a way that the prosthetic foot is supported medially and laterally on the foot shell by the heel insert positioned on its heel region.

14. The system according to claim 1, wherein at least one of the forefoot outer contour is adapted to the forefoot inner contour in such a way that the prosthetic foot is supported frontally on the foot shell by the forefoot insert positioned on its forefoot region, or the heel outer contour is adapted to the heel inner contour in such a way that the prosthetic foot is supported on the foot shell at the rear by the heel insert positioned on its heel region.

15. The system according to claim 1, wherein at least one of the forefoot outer contour, at least in sections, corresponds to the forefoot inner contour or the heel outer contour, at least in sections, corresponds to the heel inner contour.

16. The system according to claim 1, wherein at least one of the forefoot inserts or heel inserts can be at least one of attached to the respective prosthetic foot or positioned in the foot shell.

17. The system according to claim 1, wherein at least one of the heel inserts or forefoot insert exhibit different degrees of at least one of hardness, elasticity or flexibility.

18. The system according to claim 1, wherein at least one forefoot insert comprises several articulated elements.

19. The system according claim 1, wherein at least one forefoot insert includes at least one of at least one actuator, at least one deceleration element, or at least one sensor.

20. The system according to claim 1, wherein the heel inserts of the system have different heel heights.
